(19) United States
(12) Patent Application Publication
(10) Pub. No.: US 2003/0049836 A1
(43) Pub. Date: Mar. 13, 2003

(54) Cell separation system

(76) Inventor: Janette T. Phi-Wilson, Los Altos Hills, CA (US)

Correspondence Address: DORSEY & WHITNEY LLP, Four Embarcadero Center, Suite 3400, San Francisco, CA, 94111-4187, US

(21) Appl. No.: 10/219,852

(22) Filed: Aug. 14, 2002

Related U.S. Application Data
(60) Provisional application No. 60/312,482, filed on Aug. 15, 2001.

(51) Int. Cl. C12N 5/06

(52) U.S. Cl. 435/325; 435/7.21

(57) ABSTRACT

A cell separation composition and system is described. The composition includes a linker that is able to permeate the cell membranes. The linker has one end coupled to an intracellular marker that binds to molecules in target cells and an extracellular component bond to the other end and used to separate the target cells in a cell mixture.

### RELATED APPLICATIONS

This application claims priority to Provisional Application Serial No. 60/312,482 filed Aug. 15, 2001.

### FIELD OF THE INVENTION

The present invention is directed to compositions useful for separating target cells from a mixed population of cells. In particular, the compositions include a membrane permeable intracellular marker that labels target cells and an extracellular component that is employed in the separation process. Methods for using the compositions are also provided.

### BACKGROUND OF THE INVENTION

The isolation of specific cell types is often required for purposes such as biotechnology research, diagnostic testing, and treatment of various medical conditions. The isolation or separation of stem cells is highly desirable in some instances due to their ability to differentiate and proliferate into various cell lines. For example, hematopoietic stem cells (HSC) are multipotential cells that give rise to five distinct cell types: erythrocytes, granulocytes, lymphocytes, monocytes, and thrombocytes (Wheater P. R. et al. (1987). Functional Histology, Churchill Livingstone Inc., New York). In one application, these HSCs may be use in patients who need to reestablish hematopoiesis, e.g. in patients post-chemotherapy.

Cells may be separated by well-established methods, e.g. by gravitation, or by centrifugation of particles attached to target cells. However, these separation techniques are generally time-consuming. Many particle attachment processes are also mediated by antibody-coated particle binding to cell surface antigens. Fluorescent labeling methods known in the art are then used to identify and isolate the target cells. For example, a purified population of CD34+hematopoietic stem cells was described in U.S. Pat. Nos. 5,035,994 and 5,130,144 to Civin. Furthermore, a more highly purified population of hematopoietic stem cells that were CD34+, Class II HLA+, and Thy-1+ was described in U.S. Pat. No. 5,061,620 to Tsukamoto et al. However, these cell surface markers may also be found on many other lineage-committed hematopoietic stem cells (Kuby J. (1992). Immunology, W. H. Freeman and Co., New York).

Fluorescent labeling used in various immunoassays are also time-consuming. In addition, they are costly and require considerable quantities of reagents. Moreover, as the number of steps employed in such identification increases, a greater number of target entities are lost or killed. Other methodologies such as flow cytometry or fluorescent activated cell sorting may also be used for separation and, in some instances, require fewer manipulations. However, they also require expensive equipment and highly trained personnel.

Compounds that permeate the cell membrane and fluoresce upon binding of an intracellular component, e.g. an enzyme, have also been used to label and separate cells. U.S. Pat. No. 5,876,956 to Jones et al. describes the isolation of murine hematopoietic stem cells by contacting a cell mixture with a cell-permeable fluorescent aldehyde, dansylaminoacetaldehyde. WO 00/34507 to Smith et al. describes the isolation of human stem cells also using a cell-permeable fluorescent substrate, in this case BODIPY-aminoacetaldehyde (BAAA). However, both of these systems separate cells by fluorescence cell sorting techniques.

A more rapid method of cell sorting employs magnetic particles or beads. For example, U.S. Pat. No. 3,970,518 to Giaver describes the magnetic separation of cells using antibody coated magnetic particles. WO 99/37751 to Rafii et al. also describes the magnetic separation of endothelial, muscle, and neural stem cells. Furthermore, U.S. Pat. No. 4,230,685 to Senyei et al. describes a magnetically responsive microsphere having Protein A on the outer surface that separates cells, bacteria and viruses. Several other magnetic particles that may be used for cell separation are disclosed in U.S. Pat. Nos. 4,267,234 to Rembaum, 4,554,088 to Whitehead et al., and 6,228,624 to Terstappen.

However, many of the problems discusses above for non-magnetic separation systems still exist with magnetic separation systems. Therefore, new cell-separation systems that are rapid, precise and non-destructive are needed.

### SUMMARY OF THE INVENTION

The compositions of this invention and systems that utilize these compositions for cell separation include a molecule made up of a linker that is able to permeate cell membranes. The linker has a first end that is coupled, i.e. attached, to an intracellular marker, and a second end that is coupled to an extracellular component. The intracellular marker binds to an intracellular molecule to label target cells. The extracellular component permits isolation of the target cells.

The linkers are preferably lipophilic and include alkyl chains, fatty acid chains and molecules such as steroids, ethylene glycol, carbohydrate and polyethylene glycol. Amino acids may also be used as linkers. Furthermore, the linkers may also possess properties that allow their passage through cell membrane channels.

The intracellular markers that may be used for cell separation include antibodies, enzymes, enzyme substrates and fluorescent substrates. An example of a fluorescent substrate for use in this invention is BODIPY-aminoacetaldehyde. Intracellular molecules that may be bound by intracellular markers include enzymes and other cytosolic proteins and molecules that include nucleic acid or amino acid sequences.

The extracellular components that may be used for cell separation include magnetic beads such as iron/dextran beads and nickel-coated beads. Other types of extracellular components that may be used include latex beads and liposomes. Various peptides such as streptavidin or avidin, or the vitamin biotin, may also be used as extracellular components.

When isolating a target cell population from a mixed cell population, the mixed cell population is contacted with a molecule or composition including the linker that is coupled at a first end to an extracellular component, and at a second end to an intracellular marker. The linker permeates the target cell membrane so that the intracellular marker may specifically bind an intracellular molecule characteristically expressed by the target cell to label the target cell. Binding of an intracellular molecule keeps a portion of the linker within the cell and, because the intracellular marker is tethered to the extracellular component, separation techniques that separate the extracellular component will also separate the target cells from the mixture.

### BRIEF DESCRIPTION OF THE DRAWINGS

FIG. 1 is a schematic diagram of the inventive cell separation system.

### DETAILED DESCRIPTION OF THE INVENTION

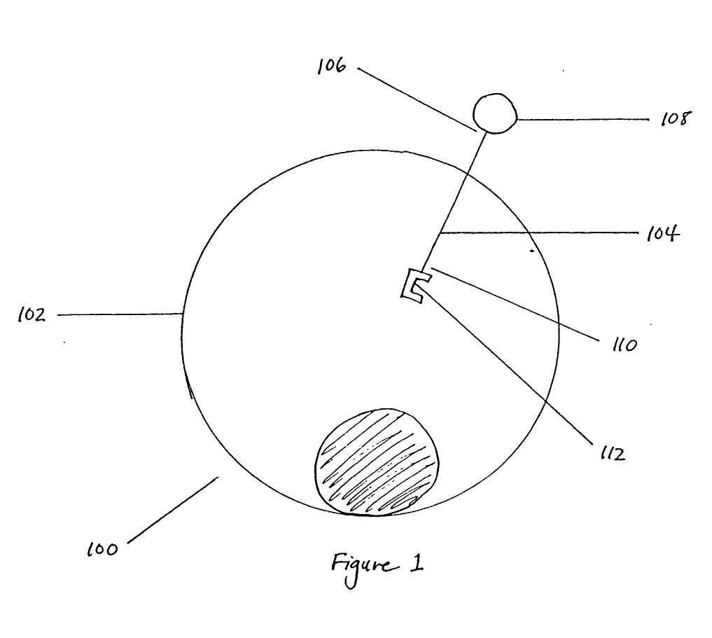

In one embodiment, target cells are labeled by contacting a cell mixture with a composition including a membrane permeable intracellular marker that binds a molecule characteristically present within the target cells. As seen in FIG. 1, target cell 100 has a cell membrane 102 that is transgressed by a linker 104. Linker 104 is coupled at a first end 106 to an extracellular component 108 and at a second end 110 to an intracellular marker 112.

Sources of cell populations that are suitable for use include umbilical cord blood, bone marrow, peripheral blood and fetal liver. Any cell population that includes stem cells can be used regardless of tissue origin. While the compositions and methods of this invention can be expected to be applicable to a variety of non-human mammalian cell populations, as well as other eukaryotic and prokaryotic cell populations, it is particularly useful in isolating human stem cells from sources including those referenced above.

### Intracellular Markers

As used herein, the term “intracellular marker” relates to any molecule or compound that binds to an intracellular molecule. For example, intracellular marker 112 may include an antibody, enzyme or enzyme substrate. Intracellular marker 112 may also be created to fluoresce when bound. Preferably, the intracellular marker is a fluorescent substrate of aldehyde dehydrogenase (ALDH). Other intracellular molecules that may be bound by an intracellular marker include DNA, mRNA and other cytosolic proteins.

### Linkers

As used herein, the term “linker” describes any compound or molecule that is coupled to an intracellular marker and allows passage of that marker through the cell membrane. In FIG. 1, linker 104 may include an alkyl chain, a fatty acid chain or an amino acid sequence. Molecules of lipids, steroids, ethylene glycol or polyethylene glycol may also be used. In general, linker 104 is lipophilic. In one embodiment, the linker passes through cell membrane channels to allow entry of the intracellular marker into the cell.

### Extracellular Components

As used herein, the term “extracellular component” describes any compound or molecule used in the process of cell separation that is coupled to a linker and remains on the outside of a target cell. Extracellular component 108 may include a magnetic bead, e.g. an iron-containing bead or nickel-containing bead, a biotin molecule, an avidin or streptavidin molecule, a latex bead or a liposome.

### Cell Separation

In use, when a mixture of cells is contacted by a composition of this invention that includes an intracellular marker, linker and an extracellular component, as described above, the intracellular marker permeates through cellular membranes due generally to the lipophilic nature of the linker. The extracellular component remains outside of the cell. Appropriate target cells are labeled by binding of the intracellular marker to an intracellular molecule, which also keeps a portion of the linker within the cell.

The separation technique used to isolate labeled or target cells from a cell mixture is dependent upon the type of extracellular component coupled to the linker. For example, if the extracellular component is a magnetic bead, target cells are directly separated by application of a magnetic field. However, if the extracellular component is a molecule such as biotin, then cell separation occurs by binding of the biotin to a solid support coated with avidin or streptavidin. In another embodiment, if the extracellular component is a non-magnetic bead, target cells may be separated from a cell mixture by centrifugation. If the target cells do not internalize a portion of the linker and bind an intracellular molecule, they are not separated out from the cell mixture.

In one embodiment, the target cell is a hematopoietic stem cell, the intracellular marker is an enzyme substrate, and the extracellular component is a magnetic particle or bead. Although all hematopoietic progenitors are known to express relatively high levels of cytosolic aldehyde dehydrogenase, human HSCs appear to express significantly higher levels than less primitive hematopoietic progenitors. In this embodiment, substrates suitable for use as the intracellular marker preferably include substrates for ALDH, particularly specific substrates for ALDH that are detectable or bear a detectable label, and that are converted by the action of ALDH to products that are detectable or bear a detectable label, and which products are retained in the target cells. In one embodiment, the substrate is a fluorescent substrate that has a discrete fluorescence emission profile similar to fluorescein isothiocyanate. An example of such a substrate is BODIPY-aminoacetaldehyde, otherwise known as BAAA.

For embodiments including magnetic beads or particles, the procedure for target cell separation preferably involves magnetic separation. In general, upon application of a magnetic field gradient, either by the placement of the cell mixture into a magnetic device, by generating a magnetic field in the container which holds the cell mixture, or by flowing the cell mixture through a flow-through device, the magnetic bead attached to the target cells will respond to the field gradient, and thus separate from the cell mixture.

The term “magnetic particle or bead” as used herein refers to any material that may or may not be permanently magnetic, which also may be paramagnetic or superparamagnetic but which in all cases exhibits a response in a magnetic field, i.e., is magnetically responsive. In one embodiment, the magnetic particles used are permanently magnetized. In another embodiment, the magnetic particles become magnetic when subjected to a magnetic field. Generally, any material which facilitates magnetic separation may be employed for this purpose.

The foregoing description of the invention is exemplary for purposes of illustration and explanation. It should be understood that various modifications may be made without departing from the scope of the invention. All documents cited above are hereby incorporated by reference in their entirety.

1. A composition for separating target cells from a mixture of cells comprising:
a linker,
an intracellular marker for binding to an intracellular molecule of target cells coupled to one end of said linker, and
an extracellular component coupled to the other end of said linker, said linker permitting the marker to penetrate the cell membrane and bind to the molecule to keep the one end portion of said linker in the cell and the other end portion and extracellular component outside of said membrane.

2. A composition as in claim 1 in which the linker is selected from the group consisting of an alkyl chain, a fatty acid chain, an amino acid sequence, molecules of lipids, steroids, ethylene glycol, carbohydrate or polyethylene glycol.

3. A composition as in claim 1 in which the marker is selected from the group consisting of an antibody, an enzyme, an enzyme substrate, a fluorescent substrate of aldehyde dehydrogenase, DNA, mRNA, or cystosolic proteins.

4. A composition as in claim 1 in which the extracellular component is selected from the group consisting of magnetic beads, iron-containing beads, nickel-containing beads, biotin molecules, avidin or streptavidin molecules, latex beads or liposomes.

5. The method of separating target cells from a mixed population of cells comprising:
contacting the cell population with a composition that includes intracellular markers, linkers and extracellular components with the markers attached to one end of the linker and the extracellular components attached to the other end of the linkers whereby the intracellular markers permeate through the cell membrane and bind to the intracellular molecules of target cells while the extracellular components remain outside of the cell, and separating the target cells on the basis of the extracellular component.

6. A method of separating target cells as in claim 5 in which the linker is selected from the group consisting of an alkyl chain, a fatty acid chain, an amino acid sequence, molecules of lipids, steroids, ethylene glycol, carbohydrate or polyethylene glycol.

7. A method of separating target cells as in claim 5 in which the marker is selected from the group consisting of an antibody, an enzyme, an enzyme substrate, a fluorescent substrate of aldehyde dehydrogenase, DNA, mRNA, or cystosolic proteins.

8. A method of separating target cells as in claim 5 in which the extracellular component is selected from the group consisting of magnetic beads, iron-containing beads, nickel-containing beads, biotin molecules, avidin or streptavidin molecules, latex beads or liposomes.
